(19) United States
(12) Patent Application Publication
(10) Pub. No.: US 2012/0132223 A1
(43) Pub. Date: May 31, 2012

(54) Compositions And Methods For Increasing Tightly Bound Water In Hair

(76) Inventors: Geoff Hawkins, Yardley, PA (US); Vasile Ionita-Manzatu, Old Bethpage, NY (US); Barbara Wolf, Scarsdale, NY (US); Lavinia Popescu, Jackson Heights, NY (US); Jack Lombardi, Massapequa, NY (US)

(21) Appl. No.: 13/201,094

(22) PCT Filed: Feb. 19, 2010
(86) PCT No.: PCT/US10/24655

§ 371 Date: Feb. 10, 2012

Related U.S. Application Data
(60) Provisional application No. 61/153,828, filed on Feb. 19, 2009.

(51) Int. Cl. A61K 8/25; A61K 8/29; C01B 33/20; A61K 8/81; A61Q 5/12; A61K 8/27; A61K 8/26

(52) U.S. Cl. 132/202; 424/70.1; 424/70.15; 423/277

(57) ABSTRACT

A hair moisturizing topical composition comprising materials that emit electromagnetic radiation at wavelengths that affect tertiary structure (breaking of disulfide bonds) in human hair, and that bring about changes in secondary structure of hair proteins. The intensity of the radiation is controlled and sufficient to cause or facilitate altering of protein structure. The invention includes methods of using such topical compositions. Testing indicates that the level of tightly bound water in hair is increased, and there is no damage to hair of the type characteristic of heat and chemical treatments.

This application claims priority of U.S. 61/153,828, filed Feb. 19, 2009, herein incorporated by reference, in its entirety.

### FIELD OF THE INVENTION

The invention is in the field of hair conditioning and protection. More particularly, it is in the field of conditioning and protecting hair by non-chemical means.

### BACKGROUND OF THE INVENTION

### Human Hair

U.S. Pat. No. 5,395,490 is herein incorporated by reference, in its entirety. FIGS. 1, 2A, 2B, 4A and 4B in U.S. Pat. No. 5,395,490 diagram the structure of human hair fibers, the protein components of hair, and energy levels of the disulfide bond.

A fiber of human hair comprises three main morphological components: the cuticle, the cortex, and the cell membrane complex, which itself is comprised of a protein matrix of keratin peptide chains, such as cysteine. A medulla may also be present. These peptide chains are linked to each other by disulfide bonds. The natural shape and structural integrity of human hair fiber depend, in part, on the orientation of the disulfide bonds which link the protein chains. The health and condition of hair also depend on the moisture content in the hair.

### Hair Under Attack

For various reasons, the hair is routinely assaulted by exposure to high heat and/or by exposure to chemicals that are reactive with hair. For example, hair may be exposed to damaging heat from a hair dryer, a flat iron curler, or the sun. Hair begins to denature at temperatures that routinely achieved by these sources, 150-250° C., for example. Hair may be intentionally exposed to damaging chemicals during straightening, perming, coloring or other cosmetic treatment, for example. Hair may also be exposed to chemicals unintentionally, as from pollution, for example. Heat or chemicals can cause hair to lose moisture.

It is well known to style the hair (i.e. straightening and curling) and color the hair, by treating the hair with chemical agents. These include, for example, treatments that use reagents to reduce and re-oxidize disulfide bonds that link protein molecules in the hair. Such reagents include mercaptans, alkalis, aldehydes, etc. These and other chemical treatments, while effective, are considered harsh and damaging to human hair. Some negative effects of hair styling include dry, brittle or limp hair; a loss of shine and/or color; damage to the scalp skin and damage to protein bonds in the hair other than the disulfide bonds. Damage to lipids in the exocuticle, swelling of the hair fiber and lifting of the cuticle also occur. Furthermore, chemical treatments are topically applied in a broad way, meaning that damage is widely distributed.

### Treatments Involving Light

The use of electromagnetic radiation to change the shape of human hair or color human hair, is also known. There are techniques that use light to directly affect the disulfide bonds that link protein molecules in the hair, and there are techniques that use light as an adjunct to other manipulations of the disulfide bonds (i.e. to accelerate one or more chemical process). Thus, “directly affect” or “direct effect” mean that a substance emits electromagnetic radiation that is absorbed by and that excites disulfide bonds, without first being absorbed by some other material.

U.S. Pat. No. 5,395,490 discloses a method of reshaping human hair by using electromagnetic radiation to rearrange disulfide bonds within the hair. Disulfide interactions are part of the hair protein's tertiary structure. During the time that the hair is exposed to the electromagnetic energy, stress is applied to the hair. As a result, once the disulfide bond is broken, each S atom is available to form a different bond with some other dissociated S atom. The structure of the new bond is determined in part by the stress. The energy required to raise an isolated disulfide bond from its ground state to the continuum (i.e. the dissociation energy) is reportedly about 2.2 eV. For a given bond that is raised to the continuum (i.e. the bond is cleaved), this energy may be supplied from a single photon or from a series of photons. There is a range of photon frequencies that may be used to cleave the disulfide bonds, however, the most efficient process takes advantage of a resonance condition. The '490 reference suggests that the energy levels of an isolated S₂ molecule lie within a frequency range of 2×10¹³ to 1×10¹⁵ Hz (corresponding to about 0.30 to 15 μm wavelength or about 0.08 to 4.13 eV). However, the '490 reference suggests that in hair, the disulfide bond is subject to other forces, and therefore a frequency range of 1×10¹³ to 2×10¹⁵ Hz (corresponding to about 0.15 to 30 μm wavelength or 0.04 to 8.3 eV) is preferred. By bombarding hair with photons in this range of resonant frequencies for a length of time, the disulfide bonds will move between their natural energy states (or modes of vibration), with some bonds being excited to the continuum state.

Nevertheless, U.S. Pat. No. 5,395,490 fails to disclose a composition that comprises a material that is able to radiate in a wavelength range around 20 μm. It fails to disclose applying the composition to the hair. It fails to disclose activating the material in the composition to radiate in a wavelength range around 20 μm. It fails to disclose methods of treating the hair, as disclosed herein. Furthermore, the '490 patent applies radiation to the disulfide bonds from complicated high and low frequency wave form generators and supporting electronics. In fact, the present invention suggests a device no more complicated than a hair dryer. Also, '490 discloses a range of photon energies 0.04 to 8.3 eV, that includes the dissociation energy of S₂, about 2.2 eV. This is unlike the present invention where a device capable of producing photons at 2.2 eV is neither required, nor preferred.

WO/1994/010873 and WO/1994/010874 disclose methods of treating hair, in particular human head hair, for cosmetic purposes. The hair is exposed to light with an intensity and wavelength chosen so that the protein structure of the hair is altered to produce the desired cosmetic effect. In WO/1994/010873 the effect is shaping hair. However, the reference discloses using light of wavelength 400 to 600 nm (0.4-0.6 μm), well below the approximately 20 μm described in the present invention. A single photon having wavelength of 400 to 600 nm “carries” about 2.05-3.0 eV of energy (which lies within the 0.04 to 8.3 eV range of the '490 patent, above). As noted, the energy required to raise a disulfide bond from its ground state to the continuum is, reportedly, about 2.2 eV. Thus, the '873 reference suggests using a narrower range of frequencies than the '490 patent, but centered around the S₂ dissociation energy. It is reasonable to expect that a wider range of frequencies disclosed in the '490 patent will be more efficient at cleaving disulfide bonds than the narrow range of frequencies disclosed in the '873 reference.

In WO/1994/010874 the cosmetic effect in view, is improved hair coloring. In particular, for the support of the chemical coloring of head hairs, light is used having a wavelength between approximately 600 nm and 1200 nm, so that a change of enzyme coordinate and/or a change of the redox potentials results. It is reported that hair coloring is improved, i.e. the colors are more brilliant than without influence of light, and less colorant is necessary than with conventional coloring. 600 to 1200 nm (0.6-1.2 μm) is well below the approximately 20 μm utilized in the present invention.

Furthermore, WO/1994/010873 and WO/1994/010874 fail to disclose a composition that comprises a material that is able to radiate in a wavelength range around 20 μm. They fail to disclose applying such a composition to the hair. They fail to disclose activating the material in the composition to radiate in a wavelength range around 20 μm. They fail to disclose methods of treating the hair, as disclosed herein. In '873 and '874, electromagnetic energy is supplied by a device; an argon laser, for example. This is unlike the present invention where a device capable of producing photons at 2.2 eV is neither required, nor preferred. Furthermore, the present invention does not require lasers and the supporting electronics to apply radiation to the disulfide bonds, as described in these patents. In fact, the present invention suggests a device no more complicated than a hair dryer.

U.S. Pat. No. 5,858,179 discloses a combination of chemicals and electromagnetic radiation used to alter the physical characteristics of keratinic fibers such as mammalian or human hair. A non-irritating, non-reactive disulfide, in the form of a solution or gel, is first contacted with the hair. Electromagnetic radiation is then applied to the hair to photo-chemically convert the disulfide into a dithiol. The dithiol breaks the disulfide bonds in the hair, so that the hair can be permanently re-shaped. U.S. Pat. No. 5,858,179 fails to disclose a composition that comprises a material that is able to radiate in a wavelength range around 20 μm. It fails to disclose applying such a composition to the hair. It fails to disclose activating the material in the composition to radiate in a wavelength range around 20 μm. It fails to disclose methods of treating the hair, as disclosed herein. U.S. Pat. No. 5,858,179 does not use electromagnetic radiation directly on the disulfide bond, to break the bond. Rather, the radiation used is chosen to convert free disulfide into dithiol using a reported wavelength of 200 to 530 nm (2.3 to 6.2 eV). Furthermore, the present invention does not require a device to generate electromagnetic radiation at specific frequencies. Rather, the present invention suggests a device no more complicated than a hair dryer.

U.S. Pat. No. 3,863,653 discloses a method and apparatus for treating fibers by enclosing them within a resonant cavity to which high frequency current is supplied, the resonant frequency and impedance of said cavity being matched to that of its supply. This method is really an adjunct to a chemical treatment method. U.S. Pat. No. 3,863,653 uses high frequency radiation to heat hair from the inside, thereby accelerating the chemical reactions and reducing the time that the hair must be exposed to the potentially damaging chemicals. The frequency of radiation disclosed is from 10-4000 MHz, wholly unsuitable for use in the present invention.

### Tourmaline

Tourmaline is an acentric rhombohedral borosilicate characterized by six-membered tetrahedral rings. It is a semi-precious stone, and a crystal silicate compounded with varying amount of elements such as aluminium, iron, magnesium, sodium, lithium, or potassium.

The compositions of tourmaline vary widely, and one general formula has been written as

XY₃Z₆(T₆O₁₈)(BO₃)₃V₃W,

where, X=Ca, Na, K, vacancy; Y=Li, Mg, Fe²⁺, Mn²⁺, Zn, Al, Cr³⁺, V³⁺, Fe³⁺, Ti⁴⁺; Z=Mg, Al, Fe³⁺, Cr³⁺, V³⁺; T=Si, Al, B; B=B, vacancy; V=OH, O; W=OH, F, O (Hawthorne and Henry 1999, Classification of the minerals of the tourmaline group. European Journal of Mineralogy, 11, 201-215).

Fourteen end-members are recognized by the International Mineralogical Association (IMA) and Hawthorne and Henry (1999) have grouped these into three principal groups, based on the dominant occupancy of the X site. These groups are the alkali group, the calcic group and the X-site vacant group. The following table with updated information is reproduced from

http://www.geol.lsu.edu/henry/Research/tourmaline/TourmalineClassification.htm.

| | | | | | | | |
|---|---|---|---|---|---|---|---|
| Species | (X) | (Y₃) | (Z₆) | T₆O₁₈ | (BO₃)₃ | V₃ | W |

Alkali tourmalines

| | | | | | | | |
|---|---|---|---|---|---|---|---|
| Elbaite | Na | Li_{1.5} Al_{1.5} | Al₆ | Si₆O₁₈ | (BO₃)₃ | (OH)₃ | (OH) |
| Schorl | Na | Fe²⁺₃ | Al₆ | Si₆O₁₈ | (BO₃)₃ | (OH)₃ | (OH) |
| Dravite | Na | Mg₃ | Al₆ | Si₆O₁₈ | (BO₃)₃ | (OH)₃ | (OH) |
| Olenite | Na | Al₃ | Al₆ | Si₆O₁₈ | (BO₃)₃ | (O)₃ | (OH) |
| Chromdravite | Na | Mg₃ | Cr₆ | Si₆O₁₈ | (BO₃)₃ | (OH)₃ | (OH) |
| Buergerite | Na | Fe³⁺₃ | Al₆ | Si₆O₁₈ | (BO₃)₃ | (O)₃ | F |
| Povondraite | Na | Fe³⁺₃ | Fe³⁺₄Mg₂ | Si₆O₁₈ | (BO₃)₃ | (OH)₃ | O |
| Vanadiumdravite | Na | Mg₃ | V₆ | Si₆O₁₈ | (BO₃)₃ | (OH)₃ | (OH) |

Calcic tourmalines

| | | | | | | | |
|---|---|---|---|---|---|---|---|
| Liddicoatite | Ca | Li2Al | Al₆ | Si₆O₁₈ | (BO₃)₃ | (OH)₃ | F |
| Uvite | Ca | Mg₃ | MgAl₅ | Si₆O₁₈ | (BO₃)₃ | (OH)₃ | F |
| Hydroxy- | Ca | Fe²⁺₃ | MgAl₅ | Si₆O₁₈ | (BO₃)₃ | (OH)₃ | (OH) |
| feruvite | | | | | | | |

X-site vacant tourmalines

| | | | | | | | |
|---|---|---|---|---|---|---|---|
| Rossmanite | — | LiAl₂ | Al₆ | Si₆O₁₈ | (BO₃)₃ | (OH)₃ | (OH) |
| Foitite | — | Fe²⁺₂Al | Al₆ | Si₆O₁₈ | (BO₃)₃ | (OH)₃ | (OH) |
| Magnesiofoitite | — | Mg₂Al | Al₆ | Si₆O₁₈ | (BO₃)₃ | (OH)₃ | (OH) |

Hawthorne and Henry (1999) also postulate at least 27 other tourmalines that have yet to be verified. Thus, in speaking of tourmaline, there are substantial differences (as well as similarities) among varieties. Some reported properties of tourmalines include: specific gravity: 2.96-3.31; index of refraction: 1.610-1.735; birefringence: 0.016-0.080; pleochroism: strong in all species; hardness: 7.0-7.5.

In terms of the present invention, performance may vary from one variety to another. In particular, emissivity and absorption spectra may vary from one variety to another. Also, the intensity of emitted radiation and the activation energy may vary from one variety to another. When used in particulate form in compositions of the present invention, these properties of tourmaline will also depend on the particle size and the concentration.

### Tourmaline-Containing Products

The use of tourmaline in hair products is known. For example, a product called IB Shield Humidity Lock-Out Shine Spray by Jonathan Product describes its use of tourmaline by saying “Tourmaline & Amethyst: Charged ionic crystal blend known to improve shine, smoothness, and manageability of hair.” Further description includes “Charged ions & Far Infrared energy help revitalize the scalp to maintain optimum hair health.”

Hai Flat Iron Fluid by Angles BeautyCare Group contains tourmaline, which the manufacturer asserts, “is claimed to deliver weightless moisture and increased absorption for beautifully conditioned hair, protect it against heat damage, reduce static, and provide longer lasting color and gorgeous shine.”

Nothing in the descriptions of these products suggests a composition that comprises a tourmaline (or any other material) that is able to radiate in a wavelength range around 20 μm, and nothing suggests activating such a material to radiate in a wavelength range around 20 μm. Even if the tourmaline does radiate in this range, nothing in the prior art suggests that the intensity is sufficient to protect hair from damage caused by heat or chemical treatment. To the best of the applicants' knowledge, in these products, as well as others, tourmaline is not reported to increase the moisture content of hair.

Tourmaline hair dryers are also known. Such hair dryers contain tourmaline crystals that deliver negative ions and far-infrared heat, which, reportedly, dries hair from the inside out. As a result, a person can dry hair faster, and the hair is left healthy and shiny with optimum manageability. Flat irons for shaping hair are also known to contain tourmaline. Typically, it is reported that the tourmaline supplies negative ions that yield softer and shinier hair, while infrared heat is associated with improved hair moisture and luster. Hair brushes and hair setting rollers with tourmaline are known. Often, the benefit associated with tourmaline is less frizz, due to an ionic effect. None of these appliances, suggests a composition that comprises a material that is able to radiate in a wavelength range around 20 μm, or at an intensity that is sufficient to protect hair from damage caused by heat or chemical treatment. To the best of the applicants' knowledge, in these products, as well as others, tourmaline is not reported to increase the moisture content of hair.

### SUMMARY OF THE INVENTION

The present invention is a topical composition that increases the moisture content of human hair. The composition comprises one or more materials that emit or are induced to emit electromagnetic radiation at specified wavelengths. The photon energies employed are well below the dissociation energy of a ground state disulfide bond. The intensity of the radiation is controlled and the process, apparently, increase the moisture content of hair. The treatment may be effective on its own or as an adjunct. The techniques disclosed herein, are non-chemical.

The invention includes compositions that may be washed out of the hair after a period of time, and compositions that are intended to remain in the hair for additional or extended benefits. The invention includes methods of using a topical composition that comprises one or more materials that emit or are induced to emit electromagnetic radiation at wavelengths that leads to an increase the moisture content of hair.

### DESCRIPTION OF THE FIGURES

FIG. 1 is a graph of the emissivity vs. wavelength of red tourmaline at 78° C.

FIG. 2 is a graph of the radiance vs. wavelength of red tourmaline at 78° C.

### DETAILED DESCRIPTION OF THE INVENTION

The present invention lies in the unexpected discovery that hair moisture may be increased, via non-chemical means, with electromagnetic energy that is supplied by a tourmaline containing topical composition. By “non-chemical” we mean that the materials in the compositions disclosed herein, do not act as reagents or catalysts with hair. By “non-chemical” we further mean that pure energy is supplied to the hair. In the present context, “topical” means applied to the surface of the hair, particularly human head hair.

The present invention also lies in the surprising discovery that certain materials can be incorporated into stable, commercially acceptable, topical hair products in quantities that are sufficient to increase the moisture of human head hair. By moisturizing the hair, the hair is protected from damaging effects, including the damaging effects of chemical and heat exposure.

Throughout the specification, “comprising” means that a collection of objects is not necessarily limited to those recited.

### Criteria for Suitable Materials and Compositions

Asking a commercially acceptable personal care composition to supply sufficient energy for significantly increasing the moisture of human hair, while remaining reasonably priced and meeting aesthetic and regulatory requirements, places a long list of requirements on the composition. It is surprising that the criteria discussed herein, could be met successfully.

a. Wavelength, Intensity, Temperature

We have observed an increase in tightly bound water in human hair, following treatment by a composition that supplies electromagnetic radiation of certain wavelengths and intensities.

For any given material that we might consider in a personal care composition, temperature is the most important factor affecting both wavelength and intensity. To a large extent, the temperature of a material determines the intensity and wavelength distribution of radiation emitted by the material. Compositions of the present invention will generally be exposed to temperatures between about 25° C. and 175° C. Therefore, a suitable material is one that, between about 25° C. and 175° C., emits electromagnetic radiation in a range of wavelengths that lead to an increase moisture content of hair. We have achieved significant, unexpected results with a range of wavelengths of about 0.15 to 30 μm. While this range of wavelengths is known to be useful to cleave disulfide bonds (tertiary structure), we have observed changes in hair secondary structure, as well. 0.15 to 30 μm covers most of the near and middle infrared. Depending on the classification, and there are several, this range may also cover a small portion (about 3%) of the far infrared, which extends to about 1000 μm wavelengths. On the other hand, some classifications suggest that the middle infrared extends up to 40 μm. The point is, that at the time of the present invention, “the boundaries between the near, mid and far-infrared regions are not agreed upon and can vary” (see “CoolCosmos Infrared Astronomy Tutorial: Near, Mid, and Far Infrared”—http://coolcosmos.ipac.caltech.edu/cosmic_classroom/ir_tutorial/irregions.html).

As note above, a suitable material of the present invention is one that, between about 25° C. and 175° C., emits electromagnetic radiation at an intensity that is useful to increase moisture in human hair. An intensity is considered “useful to increase moisture in human hair” if the hair being treated achieves a significant increase in moisture in a commercially acceptable amount of time. By “commercially acceptable amount of time” we mean less than about one hour, more preferably, less than about 30 minutes, more preferably still, less than about 10 minutes, most preferably less than about 5 minutes. This time to increase moisture is measured from the moment the composition is placed on the hair and activated. So, if an otherwise useful material would require an unacceptably long time to effect the desired change (3 hours, for example), then that material is less suitable or not at all suitable for use in the present invention, because such a product has low commercial viability.

Now, intensity (or better, radiance) of a material, is the energy per second emitted from a unit area of the material, into a unit solid angle. Radiance depends on the temperature of the material. Thus, to find a suitable material, one could begin by looking at radiance verses wavelength curves of various materials, to find those materials that have a more prominent intensity in the 0.15 to 30 μm wavelength range, when heated to the temperature range of interest, i.e. 25° C.-175° C. or 40° C.-60° C. or 60° C.-80° C. and so forth. Determining what is a useful intensity may best be done by trial and error. A candidate material may be incorporated into a base hair composition, and applied to the hair in commercially reasonable amounts. If the hair is moistened (i.e. increased in tightly bound water in the keratin structure) in a commercially acceptable amount of time, then the intensity may be considered useful.

Aside from wavelength and intensity, other parameters should be considered when attempting to identify a suitable material according to the present invention.

b. Emissivity

In U.S. Pat. No. 5,395,490 and other prior art, the source of radiation is a sophisticated electronic, multi-frequency electromagnetic wave generating device, that has its own power source. By design, most of the supplied power is converted into electromagnetic energy and the intensity of radiation at a given wavelength can be controlled to arbitrarily high precision. This is very different from the present invention, wherein the power source is the heat supplied to the suitable material (as from a hair dryer or flat iron), which is re-radiated in a wavelength-intensity spectrum that is characteristic of the material. The input power is limited to what is safely supplied by a generic consumer hair dryer or flat iron. Thus, not having an essentially unlimited supply of power, it is important that a suitable material be relatively efficient at re-radiating the energy that it absorbs so that the intensity will be useful. Thus, in addition to looking at the radiance of a potential suitable material, one should also look at the emissivity (a measure of a material's ability to radiate the energy that the material has absorbed). An example of an inefficient material for the present invention is one that radiates at a suitable wavelength, but the amount of material needed to increase moisture in the hair is commercially and/or cosmetically infeasible.

Like radiance, the emissivity of a material also depends on the temperature of the material. Thus, in addition to radiance verses wavelength, one could look at emissivity verses wavelength curves to find materials in the 0.15 to 30 μm wavelength range that have high emissivity, in a temperature range of interest, i.e. 25° C.-175° C. or 40° C.-60° C. or 60° C.-80° C. and so forth. Suitable materials have emissivity greater than about 0.50. Preferred materials have an emissivity greater than about 0.80. Materials most preferred have emissivity greater than about 0.90.

Thus, initial requirements for a suitable material include: one that that has emissivity greater than about 0.50, so that when heated to 25° C.-175° C. the material emits in the 0.15 to 30 μm wavelength range, at an intensity that leads to an increase in moisture in human hair, in a commercially acceptable amount of time. It was wholly unclear that such a material should exist or that human hair could be moistened by radiation coming from a material that is activated with heat. This is because, in general, we think of heat and radiation as damaging and drying to human hair.

c. Commercial Considerations

To be a suitable material, we must be able to use the material in quantities that are commercially reasonable for use in a cosmetic product, while still being effective. What is “commercially reasonable” depends on cost, manufacturing difficulties, ability to stabilize the composition, look, feel, smell and overall impression of the composition, etc. So, for example, if an otherwise useful material imparts a foul odor to the composition in which it is disposed, then that material is less suitable or not at all suitable. Or if an otherwise useful material destabilizes the composition in which it is disposed, then that material is less suitable or not at all suitable. A person of ordinary skill in the art is able to identify a composition of unacceptable consumer quality or low commercial viability, and is thus able to steer clear of materials that are not commercially reasonable.

Furthermore, a suitable material is one that is suitable for use in cosmetic preparations, from a safety standpoint; at a minimum meeting all relevant controlling regulations for cosmetic products. So, if an otherwise useful material is banned by all or some regulatory authorities, then that material is less suitable or not at all suitable, because a commercial product cannot be achieved. It was surprising that material(s) meeting all of the physical, formulary and commercial requirements herein discussed, could be found.

d. Activation/Deactivation of the Suitable Material

Furthermore, a preferred suitable material is one that must be activated before it will significantly affect the tightly bound moisture content of human hair, and which can be deactivated to stop the effect. It is realized that many materials, even at room temperature, emit some radiation in the 0.15 to 30 μm wavelength range. However, by “activated”, we mean that the intensity of radiation emitted by the suitable material is “useful to increase moisture in human hair” in a “commercially acceptable amount of time”. Thus, if a suitable material is emitting radiation in the 0.15 to 30 μm wavelength range, but the intensity is such that significant moistening does not occur within about twenty-four hours, more preferably within about 12 hours, even more preferably within about 1 hour, and most preferably within about 30 minutes, then that material is not “activated” as herein defined.

Preferred methods of activation and deactivation must be suitable for consumer use and be commercially sensible in the personal care market. So, for example, if an otherwise useful material requires an activation/deactivation that is inconvenient from a consumer standpoint or that requires copious amounts of energy, then that material may not be suitable. A preferred activation method is heating with a hair dryer, either a handheld hair dryer or a commercial hair dryer typically found in hair salons. This method of activation is preferred, because it is already expected that compositions according to the present invention will be subjected to heat from a hair dryer or hair shaping tool, as the consumer goes about her usual grooming or beauty routine. Accordingly, a preferred suitable material is one that does not produce effective wavelengths and/or intensity, until the material is heated to 40° C. to 60° C., more preferably above 80° C., and most preferably between 60° C. and 80° C. A minimum of 40° C. is useful to prevent unwanted activation of the composition. Temperatures above 80° C. can be used to activate the suitable material, but the temperature itself begins to have a detrimental effect on the hair. Therefore, the most preferred activation temperatures are between about 60° C. and 80° C. These temperatures are achievable with a handheld hair dryer, even though the source of hot air is several inches from the hair and the hot air flow may not be continuously directed on the same portion of hair. Preferably, activation is achievable within ten minutes of blow drying, more preferably, within five minutes of blow drying, most preferably, within one minute of blow drying. We note that devices other than hair dryers may be used; for example flat irons. However, if a flat iron is used, it is preferably used to heat the suitable material to it's most preferred temperature, and no more, thereby limiting any damage or drying from excessive heat.

We also expect that activation, depending on the emitting material, may be achievable by light. In this embodiment, shining a visible light (red, blue, green etc) on the suitable material causes the suitable material to radiate in the 0.15 to 30 μm wavelength range. The intensity of the emitted radiation, in general, depends on the intensity of the visible light activation source. But we expect that an effective and commercially viable combination of visible source light and suitable radiating material may be found. Deactivation is achieved by removing the visible light source. Activation and deactivation by this method would be essentially immediate, since there is no waiting for the suitable material to heat up.

### Tourmalines as Suitable Materials

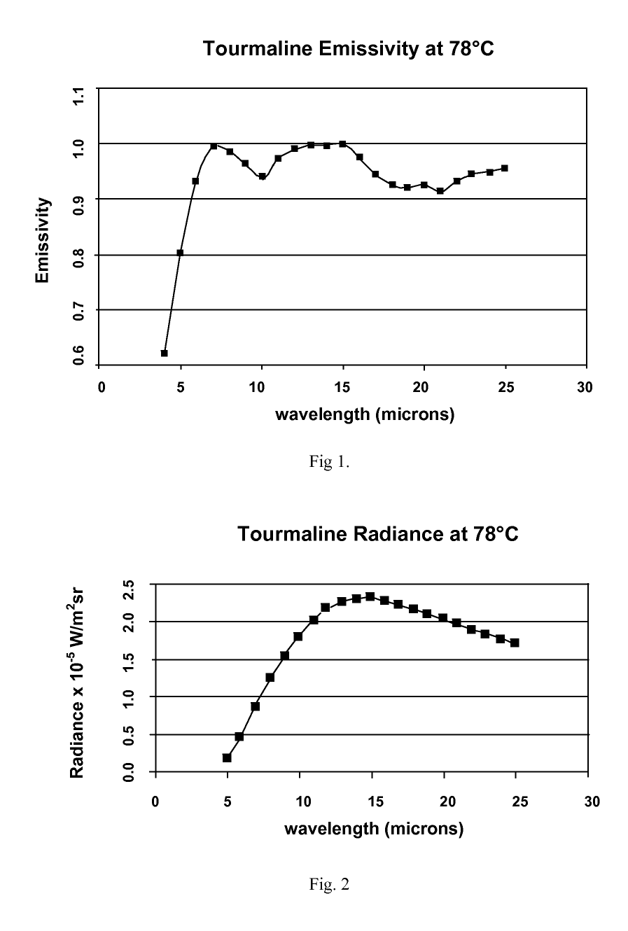

Unexpectedly, we have discovered that tourmalines are very useful in the compositions of the present invention. Referring to FIG. 1, this particular red tourmaline, heated to 78° C., has emissivity well over 0.9 in the wavelength range with which we are concerned. At 20 μm wavelength, the emissivity is about 0.93. Though not shown, the emissivity of this material, at 20 μm, drops to about 0.75 when the temperature is reduced to about 44° C.

Referring to FIG. 2, the energy output of this particular red tourmaline, heated to 78° C., peaks between about 10 and 20 μm wavelength. 78° C. is a temperature that is not unusual when styling the hair.

But having identified a material (red tourmaline, for example) with the right wavelength and high emissivity, the question remained, is the intensity sufficient to make a commercial product. In other words, what surface area of red tourmaline will emit enough energy to effectively increase moisture in human hair, in a commercially acceptable amount of time? Also, could that surface area be achieved in an amount by weight of tourmaline that can be incorporated into a commercially viable product? We have discovered that the answer to both questions is yes. We have demonstrated, for the first time, that increasing the levels of tightly bound water in human head hair can be achieved by using a topical composition comprising one or more tourmalines. This treatment is considered non-chemical. By “non-chemical” we differentiate from known commercial treatments that interact with hair through molecular interactions, rather than photon absorption.

Surprisingly, tourmaline compositions that are safe, stable and commercially acceptable, as well as effective, were achieved. The tourmaline is used in quantities that are reasonable for commercial cosmetic products, and yet the tourmaline still supplies enough electromagnetic energy to effect an increase in keratin moisture. The tourmaline must be activated before it will significantly affect the hair, and can be deactivated to stop additional effect.

In another embodiment the activation of tourmaline is achieved by shining a visible light on the tourmaline. For example, we note that red and pink tourmalines have absorption lines at 458 and 451 nm, as well as a broad absorption band in the green spectrum. Blue and green tourmalines have a strong, narrow absorption band at 498 nm and almost complete absorption of red, down to 640 nm. In turn, these materials re-emit a portion of the incident light energy in the 0.15 to 30 μm wavelength range, and therefore, may be useful to increase moisture in human hair. Suitable sources of visible light include LEDs and lasers. With these devices, the light can be concentrated and directed.

### DSC Analysis

Protein denaturation is a process in which proteins lose their secondary, tertiary or quaternary structure by application of some external stress or compound, such as a strong acid or base, a concentrated inorganic salt, an organic solvent (e.g., alcohol or chloroform), or heat, but the peptide bonds between the amino acids (primary structure) are left intact. Denaturation of tertiary structure includes disruption of interactions between amino side chains, such as covalent disulfide bridges between cysteine groups, non-covalent dipole-dipole interactions between polar groups, and Van der Waals interactions between non-polar groups in the side chains. Denaturation of secondary structure means that proteins lose all regular repeating patterns (such as alpha-helix structure and beta-pleated sheets), and adopt a random coil configuration. Thermal denaturation of the helical keratin fraction in hair occurs at about 210 to 260° C.

In the literature, loosely bound water in hair has been described as that water which is driven out of hair starting at about 100° C., to about 140° C., resulting in an endothermic transition readily seen in the DSC thermogram. Strongly bound water has been described as that which is driven out of hair at approximately 220° C. to about 260° C. DSC is a thermal analysis technique used to measure transition temperature and heat of transformation (enthalpy) for endothermic (heat generator) and exothermic (exhaust of heat) reactions. DSC is typically used to measure melting and solidification temperatures at different melting or cooling rates. It is reported that keratin moisture content and protein denaturation can be detected by differential scanning calorimetry.

Moisture content measurements were made on untreated hair (control), a base formula (no tourmaline), 5% red tourmaline in the base formula, and MIZANI® Rhelaxer, a commercially available sodium hydroxide hair straightening conditioning product. The hair sample treated with 5% tourmaline, following application of the tourmaline composition to the hair, the hair was subjected to blow drying. Samples were prepared for DCS by placing small cut pieces (2-10 mg) of hair into a 50 μl aluminum pans, then hermetically sealing each pan with an aluminum lid and crimping tool. A Perkin Elmer Pyris 1 DSC was programmed to perform the following thermal profile: stabilize at 25 C for 2 minutes, heat at 10 C/minute to 260 C, end test and return to 25 C. Endothermic calculations were performed by identifying the beginning temperature of the transition and the end temperature of the transition. The area under each transition curve (enthalpy) was calculated based on the sample weight and energy required during the transition. Transition temperature peak and transition onset temperature are generated during the enthalpy calculation. Results were as follows:

| | Loosely Bound | Tightly Bound |
|---|---|---|
| Treatment | Water Energy ΔH | Water Energy ΔH |
| Control | 114.2 J/gm | 6.31 J/gm |
| Base Formula | 75 J/gm | 7.5 J/gm |
| MIZANI ® | 108 J/gm | 6.3 J/gm |
| Rhelaxer | | |
| 5% Tourmaline in | 103 J/gm | 8.32 J/gm |
| Base with blow dry | | |

The results show that treatment with a 5% tourmaline composition and blow dry, increases the tightly bound water in the hair keratin, compared to the control (about a 32% increase, only some of which may be attributed to the base formula). In contrast, as a result of the Mizani® treatment, tightly bound water remained the same as in the control. Thus, we have discovered that hair treated by an activated tourmaline composition is effective to increase tightly bound water in hair.

### Dimensions of Hair

We also measured changes in fiber dimensions, as a result if various treatments. Four groups of sixty hair fibers each, were prepared as follows. One group was a control, washed in Bumble and Bumble Alojoba Shampoo, rinsed and blown dry; one group was treated with MIZANI® Rhelaxer, 0.3 mL applied to each fiber; one group was treated with 2% red tourmaline in a cream base (see formula 1), 0.2 mL applied to each fiber, followed by blow dryer heating for about 5 minutes; one group was treated with 2% red tourmaline in a gel base (see formula 2), 0.2 mL applied to each fiber, followed by blow dryer heating for about 5 minutes.

Formula 1 - 2% Red Tourmaline Cream

| | Percent by weight |
|---|---|
| Ingredients | of composition |
| purified water | 67.70 |
| Aristoflex ® AVC (Ammonium | 1.00 |
| Acrylodimethyltaurate/VP | |
| Copolymer) | |
| glycerine | 2.00 |
| phenoxyethanol | 0.70 |
| Polyvinylpyrrolidone (PVP) | 3.00 |
| glycerin/water/sodium PCA/ | 5.00 |
| urea/trehalose/polyquaternium- | |
| 51/sodium hyaluronate | |
| cetearyl alcohol/cetearyl glucoside | 4.60 |
| PEG-100 stearate | 1.00 |
| cetyl alcohol | 2.00 |
| petrolatum | 3.00 |
| shea butter | 5.00 |
| polyquaternium-7 | 2.50 |
| red tourmaline | 2.00 |
| caprylyl glycol/phenoxyethanol/ | 0.50 |
| hexylene glycol | |

Formula 2 - 2% Red Tourmaline Gel

| | Percent by weight |
|---|---|
| Ingredients | of composition |
| purified water | 86.30 |
| carbomer | 1.00 |
| glycerine | 2.00 |
| phenoxyethanol | 0.70 |
| Polyvinylpyrrolidone (PVP) | 3.00 |
| glycerin/water/sodium PCA/ | 5.00 |
| urea/trehalose/polyquaternium- | |
| 51/sodium hyaluronate | |
| red tourmaline | 2.00 |

The fibers were then equilibrated to 80% relative humidity. The cross sectional diameters were measured, using the Fibre Dimensional System (FDAS765-Dia-Stron). Pre and post treatment values for mean cross sectional are given in the table below.

| | | | | |
|---|---|---|---|---|
| Treatment. | Mean | +SD | T test | Significance |

Control

| | | | | |
|---|---|---|---|---|
| Initial | 5323.6 | 1089.2 | | |
| Post treatment | 5452 | 1063 | 0.65 | NS |

2% Tourmaline gel

| | | | | |
|---|---|---|---|---|
| Initial | 5405 | 1036 | | |
| Post treatment | 8705 | 2076 | 11.01 | p < 0.001 |

2% tourmaline cream

| | | | | |
|---|---|---|---|---|
| Initial | 4915 | 859 | | |
| Post treatment | 7612 | 1800 | 10.47 | p < 0.001 |

Mizani ® product

| | | | | |
|---|---|---|---|---|
| Initial | 5067 | 769 | | |
| Post treatment | 5140 | 807 | 0.50 | NS |

The control demonstrated no significant difference in diameter following treatment. The 2% tourmaline gel gave a 61% increase in diameter and this was highly significant at p<0.001. The 2% tourmaline cream gave a 55% increase in diameter, which was also significant at p<0.001. In contrast, the Mizani® product did not increase the diameter by a significant amount.

### X-Ray Scattering

Wide angle x-ray scattering (WAXS) and small angle x-ray scattering (SAXS) were used to determine protein keratin structure of hair fibers after various treatments.

Five samples were prepared. A control sample was not treated; one sample was treated with a base formula and blown dry; one sample was treated with 2% tourmaline in the base formula and blown dry; one sample was treated with 2% NaOH (pH=13.45) and blown dry; one sample was treated with 4% urea in the base formula (pH=7) and blown dry;

The WAXS data provides information about secondary protein keratin structures such as: alpha, beta, alpha+beta, etc. of the hair fiber, where as the SAXS data provides information about longitudinal distance structure in the hair fiber between 1-100 nm such as coiled-coil, amorphous, ordered glycoprotein molecules, etc. For the analysis that follows, “strong” is defined as a dominant/sharp protein structure of the sample, “weak” is defined as existing/broad/vague protein structure, and “absent” means the structure is not present in the hair fiber. “Appearing” is defined as the space where the x-ray measured the protein structure. In 2-dimensional x-ray scattering images, we were able to clearly distinguish strong, weak and absent protein structures. These show up as strong, weak or absent reflections (arc/dot/ring), at certain values of the scattering vector q. The results, including q values, are listed in the next two tables.

| | | | .98 nm* |
|---|---|---|---|
| | .51 nm* | .465 nm* | Alpha & beta |
| Treatment | Alpha keratin | Beta keratin | keratin |
| Control | Strong, appear at | Weak, appear at | Weak, appear at |
| | 0.58 nm | 0.51 nm | 1.20 nm |
| | (q = 1.08 A⁻¹) | (q = 1.23 A⁻¹) | (q = 0.52 A⁻¹) |
| Base formula | Strong, appear at | Absent | Absent |
| | 0.58 nm | | |
| | (q = 1.08 A⁻¹) | | |
| 2% Tourmaline | Strong, appear at | Strong, appear at | Strong, appear at |
| | 0.56 nm | 0.48 nm | 0.97 nm |
| | (q = 1.12 A⁻¹) | (q = 1.31 A⁻¹) | (q = 0.64 A⁻¹) |
| 2% NaOH | Absent | Weak, appear at | Weak, appear at |
| | | 0.52 nm | 1.16 nm |
| | | (q = 1.21 A⁻¹) | (q = 0.54 A⁻¹) |
| 4% Urea | Strong, appear at | Strong, appear at | Weak, appear at |
| | 0.57 nm | 0.48 nm | 1.20 nm |
| | (q = 1.10 A⁻¹) | (q = 1.31 A⁻¹) | (q = 0.52 A⁻¹) |

WAXS Data: q = x(A⁻¹), scattering vector = 4πsinθ/λ, where λ is x-ray wavelength and scattering angle 2θ.

| | Meridional | | | |
|---|---|---|---|---|
| | reflection | Reflection | Reflection | Reflection |
| Treatment | 6.7 nm^{(a)} | 5.8 nm | 4.65 nm^{(b)} | 4.0 nm |
| Base formula | Absent | Weak | Absent | Strong, appear at |
| | | | | 4.0 nm |
| | | | | (q = 1.57 nm⁻¹) |
| 2% Tourmaline | Strong, appear at | Strong, appear | Weak, appear | Strong, appear at |
| | 6.7 nm | at 5.8 nm | at 4.7 nm | 4.0 nm |
| | (q = 0.94 nm⁻¹) | (q = 1.08 nm⁻¹) | (q = 1.34 nm⁻¹) | (q = 1.57 nm⁻¹) |
| 2% NaOH | Absent | Absent | Absent | Absent |
| 4% Urea | Strong, appear at | Weak, appear at | Strong, appear | Weak, appear at |
| | 6.7 | 5.8 nm | at 4.7 nm | 4.0 nm |
| | (q = 0.94 nm⁻¹) | (q = 1.08 nm⁻¹) | (q = 1.34 nm⁻¹) | (q = 1.57 nm⁻¹) |
| Control | Weak, appear at | Absent | Absent | Weak, appear at |
| | 6.7 nm | | | 4.0 nm |
| | (q = 0.94 nm⁻¹) | | | (q = 1.57 nm⁻¹) |

SAXS Data: q = x(nm⁻¹), scattering vector = 4πsinθ/λ, where λ is x-ray wavelength and scattering angle 2θ.
Notes:
^{(a)}6.7 nm sharp meridional reflection is determined by the coiled-coil keratin structure; 6.7 is the seventh order of the main period.
^{(b)}4.65 nm reflection is interpreted as arising from the order of the glycoprotein moleculars ordered in the liquid-crystalline structure, which is related to the flexible ECM.

The control sample has a strong alpha keratin structure, weak beta structure, and a weak alpha+beta structure. It has a coiled-coil structure. It should be noted that WAXS shows a very weak peak at 0.40 nm for this sample, however its protein structure is not identified, and could be due to non-homogeneous hair structure. The SAXS data shows that the control sample has the 6.7 nm meridional reflection, which corresponds to the coil-coil keratin structure.

The base formula sample has a strong alpha keratin structure, with two signature features in the wide angle x-ray scattering region: (1) a broad equatorial spot centered at 1.15 nm, corresponding to the mean distance, or spacing, between alpha helical axes, and (2) a fine meridian arc at 0.58 nm, which is related to the projection of the alpha helical pitch along the coiled-coil axis, above a broader arc around 0.57 nm of less ordered coiled coil. The sample has no coiled-coil structure and no ordered glycoprotein moleculars. The SAXS data shows structures at 5.8 nm and 4.0 nm for sample 0, however their shapes are not identified. This could be due to non-homogeneous hair structure.

The WAXS data shows that the 2% tourmaline sample has a strong alpha structure, a strong beta structure, and a strong alpha+beta structure. The SAXS data points to both coiled-coil structure and ordered glycoprotein moleculars.

The 2% NaOH sample is different from the others. It has no alpha keratin structure, no meridian arc around 1.58 nm, and no equatorial spots. A weak beta structure (equatorial arc at 0.52 nm), and a weak alpha+beta structure are detected. It should be noted that WAXS shows a very weak peak at 0.36 nm for Sample 2, however its protein structure is not identified, and could be due to non-homogeneous hair structure. The SAXS data shows that sample 2 has no ordered molecular protein structure.

The 4% urea sample has a strong alpha structure, a strong beta structure, and a weak alpha+beta structure. It has both coiled-coil structure and a 4.7 nm peak (ordered glycoprotein moleculars) that is more pronounced than that of the 2% tourmaline sample.

Based on the WAXS data, the effect of the 2% tourmaline treatment seems to be the development of a strong beta structure and a strong alpha+beta structure.

Based on the SAXS data, the effect of the 2% tourmaline treatment seems to be the development of coiled-coil structure and ordered glycoprotein moleculars.

It may seem unlikely that the formation of new secondary structure could not be accounted by the breaking and/or rearranging of disulfide bonds in the hair, because disulfide interactions are a tertiary structure. But this may not be the case. For example, it may be only after a sufficient number of disulfide bonds are broken, that some other agent is able to influence the secondary structure. At any rate, we have definitively observed that activated tourmaline cleaves disulfide bonds, enhances secondary structure, and increases the tightly bond moisture content of human keratin. Not wishing to be bound by any on theory, the development of additional secondary structure, may well explain at least some of the increased moisture content, observed above. The additional structure may provide more opportunity to retain water.

### Other Suitable Materials

Only following these proof of concept tests, has it become clear that materials other than red tourmaline are likely to be useful in the present invention. For example, various other tourmalines (i.e. black, green, pink, brown, blue) are expected to be similarly useful as red tourmaline. Also useful may be various ceramics and non-metals that emit radiation in the near and middle infrared, and that have emissivities above 90% at the working temperatures described herein. Graphite, gypsum and clays may be examples of useful non-metals. Any candidate material must satisfy the criteria discussed above.

### Compositions

Compositions of the present invention must satisfy certain criteria. For example, the compositions must be cosmetically acceptable and commercially viable. “Cosmetically acceptable” and commercially viable” or the like, usually imply that a composition is stable under typical conditions of manufacture, distribution and consumer use. By “stable”, we mean that one or more characteristics of a personal care composition do not deteriorate to an unacceptable level within some minimum period of time after manufacture. Preferably, that minimum time is six months from manufacture, more preferably one year from manufacture, and most preferably more than two years from manufacture.

An efficacious composition according to the present invention includes a composition that emits or is induced to emit photons at an intensity and range of wavelengths that leads to an increase in water in human hair. Compositions of the present invention must be efficacious when used in reasonable amounts. A composition is considered efficacious, only if the amount of composition applied to the hair is what a consumer would consider reasonable. For example, if a lotion composition increases water in hair, but a gallon of the composition is required, then this is not an effective composition according to the present invention. A person skilled in the art of personal care hair products has a very good idea of what consumers would consider reasonable. The amount of a composition of the present invention required for one treatment depends on the type and amount of hair being treated. However, experience suggests that preferably, about 5 ounces or less of a composition according to the present invention is effective to complete a treatment of a full head of hair; more preferably, about 2.0 ounces or less; most preferably, about 1.0 ounce or less. While these amounts are preferred for commercial and consumer reasons, the present invention also contemplates larger amounts, as the case may necessitate.

Within the guidelines, herein discussed, virtually any cosmetically acceptable or commercially viable composition, that is beneficial or benign to human hair, can serve as a base composition. Generally, one could say that the base composition should not absorb too much of the radiation emitted by the suitable material, and the base composition should not interfere with activation or deactivation of the suitable material. With those restrictions, a composition according to the present invention may contain any ingredients that are known to provide a benefit to the hair, any ingredients required to render a stable product, and any ingredients that render the product more cosmetically acceptable or commercially viable.

Compositions according to the present invention may contain chemical moisturizing agents as an adjunct to the non-chemical mechanism disclosed herein. Preferably, however, a composition according to the present invention has no chemical moisturizing agents, as these may have other unwanted or unanticipated effects. Preferably, the only mechanism of increasing the water content of hair, is by exposure to electromagnetic radiation supplied from the tourmaline or other suitable material in the composition.

Compositions according to the present invention may advantageously contain chemical hair coloring agents or chemical hair shaping agents. Chemical hair coloring and hair shaping reactions of the type well known in the art, tend to damage and dry hair, so use of the techniques herein disclosed is expected to counteract that damage.

The composition may have virtually any form, even solid or semi-solid, provided the composition can be distributed throughout the section of hair being treated, and along its length, from root to tip.

The suitable material may be added to the base composition or added during the manufacture of the base composition in any manner that the circumstances may require or allow. Some suitable materials may be incorporated into the composition by simple mixing, others may require pretreatments. The composition may be a mixture, a suspension, emulsion, a solid, a liquid, an aerosol, a gel, or mousse, just to name a few. The composition may be in the form of shampoo or conditioner. The composition may be hydrous or substantially anhydrous. “Substantially anhydrous” means less than about 10% total water content.

Tourmalines are expected to be useful at concentrations as low as about 1%. Regarding upper limits, in general, there may practical upper limits to the concentration of tourmaline or other suitable material. However, the practical upper limit of any particular suitable material depends on many factors, not the least of which is how much product does a consumer apply, expecting to get a certain result. Thus, in a commercial product, trial and error or consumer use testing may be the best way to determine the concentration of the suitable material. An example of a controlled trial and error experiment might be, strengthening hair samples with a defined amount of compositions comprising increasing concentrations of a suitable material, and observing the concentration at which no additional benefit is derived. The defined amount should be based on market knowledge of how much product consumers are likely to use for the given amount and type of hair. Useful compositions will contain up to about 1% of one or more tourmalines, preferably up to about 2% of one or more tourmalines, and more preferably up to about 5% of one or more tourmalines. Tourmalines are expected to be useful at concentrations up to at least about 10% of the composition. Other, more efficient emitter materials (higher emissivity) may be useful at concentrations well below 1%, while less efficient materials (lower emissivity) may only be useful at higher concentrations; above about 5% for example, or even above about 10%, for example.

Formula 3 is an example of a cosmetically acceptable, commercially viable, effective composition according to the present invention, containing 5% tourmaline.

Formula 3 - 5% Red Tourmaline Cream

| | Percent by weight |
|---|---|
| Ingredients | of composition |
| purified water | 65.20 |
| Aristoflex ® AVC (Ammonium | 1.00 |
| Acrylodimethyltaurate/VP | |
| Copolymer) | |
| glycerine | 2.00 |
| phenoxyethanol | 0.70 |
| Polyvinylpyrrolidone (PVP) | 3.00 |
| cetearyl alcohol | 4.60 |
| PEG-100 stearate | 1.00 |
| cetyl alcohol | 2.00 |
| petrolatum | 3.00 |
| shea butter | 5.00 |
| polyquaternium-7 | 2.50 |
| red tourmaline | 5.00 |
| glycerin/water/sodium PCA/ | 5.00 |
| urea/trehalose/polyquaternium- | |
| 51/sodium hyaluronate | |

Procedure:

SEQ 1: In a main kettle add water and Aristoflex®. Mixed at room temperature until clear and uniform. Continue mixing and slowly add glycerine, phenoxyethanol, PVP, and glycerin/water/sodium PCA/urea/trehalose/polyquaternium-51/sodium hyaluronate. Start to increase the temperature to 70-75° C.

SEQ 2: In a separate kettle add cetearyl alcohol, PEG-100 stearate, cetyl alcohol, petrolatum, and shea butter. Increase the temperature to 75° C., and mix until the solution is clear.

Add SEQ 2 to SEQ 1, and reduce the temperature to 40-45° C. Continue mixing, and add polyquaternium-7, and red tourmaline. Continue mixing and cool to room temperature.

### Methods

The present invention includes methods of using the compositions, herein described. A basic method includes providing a composition according to the present invention; activating the composition to emit the photons; and causing the photons to be directly absorbed by the disulfide bonds in the hair. The amount of composition applied is preferably about 5 ounces or less, more preferably about 2 ounces or less, and most preferably about one ounce or less. The step of applying the composition includes distributing the composition throughout the section of hair being treated, and along its length, from root to tip. The step of activation may include directing a flow of hot air at the section of hair for a time sufficient to activate the composition. Alternatively, the step of activation may include irradiating the section of hair with visible light, as from an LED or laser. Methods may include washing the hair before or after treatment. Methods may include repeating application to the same section of hair or using an adjunct treatment on the same section of hair.

The idea of a commercially viable, topically applied, safe and stable composition that protects and increases the water content in hair, via heat activated radiation, is new and non-obvious. The results achieved were unexpected and unlike anything in the prior art. The hair is not subjected to harsh chemicals and no mal-odor occurs. Novelty and non-obviousness are partly demonstrated by the following facts: this is the first time that this problem has been identified; this description is the first disclosure of a list of criteria that a solution to the problem must satisfy; this is the first time that a composition that meets those criteria has been disclosed. In other words, we identified the problem, found some solutions, and also defined criteria for all other solutions to the problem.

1. A topical hair composition that emits or is induced to emit photons at an intensity and range of wavelengths that are effective to increase the tightly bound water content of human hair, wherein the range of wavelengths is between 0.15 and 30 μm.

2. (canceled)

3. The composition of claim 1 comprising a material that has an emissivity of at least 0.80, in the 0.15 and 30 μm wavelength range, when the material is heated to 40° C. to 80° C.

4. The composition of claim 3 wherein the material is a tourmaline.

5. The composition of claim 4 comprising 1% to 10% tourmaline.

6. A composition according to claim 1 that is safe, stable and commercially viable from a consumer perspective.

7. The composition of claim 5 further comprising one or more film formers.

8. The composition of claim 7 comprising one or more polyvinylpyrrolidone-based film formers.

9. A method of increasing tightly bound water content of human hair, comprising the steps of:
providing a portion of a composition according to claim 1;
activating the composition to emit the photons; and
causing the photons to be directly absorbed by the disulfide bonds in the hair.

10. The method of claim 9 wherein the portion of composition is about 2 ounces or less.

11. The method of claim 9 wherein the step of activation includes heating the section of hair to at least 40° C.

12. The method of claim 11 wherein the step of activation includes heating the section of hair to at least 60° C.

13. The method of claim 11 wherein the step of heating includes directing a flow of hot air at the section of hair for a time sufficient to activate the composition and increase the tightly bound water content of human hair.

14. The method of claim 13 wherein the flow of hot air is supplied by a hair dryer.

15. The method of claim 13 wherein the sufficient time is less than about 30 minutes.

16. The method of claim 15 wherein the sufficient time is less than about 10 minutes.
